(19) United States
(12) Patent Application Publication
(10) Pub. No.: US 2022/0087597 A1
(43) Pub. Date: Mar. 24, 2022

(54) METHOD AND SYSTEM FOR ASSESSING THE STATE OF HEALING OF A FRACTURED LONG BONE

(71) Applicants: MONASH UNIVERSITY, Clayton, Victoria (AU); ALFRED HEALTH, Melbourne, Victoria (AU)

(72) Inventors: Matthias Russ, Melbourne, Victoria (AU); Mark Fitzgerald, Melbourne, Victoria (AU); Wing Kong Chiu, Clayton, Victoria (AU)

(21) Appl. No.: 17/420,098

(22) PCT Filed: Dec. 24, 2019
(86) PCT No.: PCT/AU2019/051434

§ 371 Date: Jun. 30, 2021

(30) Foreign Application Priority Data
Jan. 3, 2019 (AU) 2019900018

(51) Int. Cl. A61B 5/00; A61B 5/11

(52) U.S. Cl. CPC A61B 5/4504; A61B 5/0057; A61B 2562/0219; A61B 5/742; A61B 5/7235; A61B 5/1126

(57) ABSTRACT

A method of assessing the state of healing of a fractured long bone in a limb, including the steps of: applying a known force to the limb; using vibration sensors attached on either side of the limb to produce output signals generated in response to the known force from the output signals of the vibration sensors; from the output signals, generating frequency domain waveforms for a) phase difference between vibration sensor output signals, b) coherence of the vibration sensor output signals, and c) cross-spectra of the vibration sensor output signals; identifying in-phase and the out-of-phase responses of the vibration sensors from phase differences in the phase difference waveform at frequencies corresponding to peaks in the cross-spectra waveform; verifying coherent modes from the magnitude of the coherence waveform; and generating bone healing data, including using the magnitude of the coherence waveform and the phase differences as weighting-functions, computing a healing index value representing the state of healing of the bone.

### TECHNICAL FIELD

The present invention relates generally to a method and system for assessing the state of healing of a fractured long bone. The invention is suitable for use in applications in which an internal fixation has been applied to the long bone to assist healing, and it will be convenient to describe the invention in relation to that exemplary but non-limiting application.

### BACKGROUND OF INVENTION

“Long bones”, that is bones that are longer than they are wide, include the femur (the longest bone in the body), as well as relatively small bones such as those found in fingers. Long bones function to support the weight of the body and facilitate movement. Long bones are mostly located in the skeleton and include bones in the lower limbs (tibia, fibula, femur, metatarsals and phalanges) and bones in the upper limbs (the humorous, radius, ulna, metacarpals and phalanges).

Internal fixations are a common treatment for a fractured long bone to correct alignment, provide mechanical stability, allow weight bearing and prompt early use of the limb while the bone is healing. Internal fixation allows patients to return to normal function earlier than casts and splints allow, as well as reducing the incidents of non-union and mal-union of the bone. FIG. 1 show an example of an installed plate 10 and associated screws, such as the screw referenced 12, providing internal fixation to a long bone 14. Similarly, FIG. 2 shows an example of an intramedullary rod, also known as an intramedullary nail (IM nail), including a metal rod 16 forced into the medullary cavity of a bone and associated screws 18.

An essential part of the treatment is accurately determining healing progression and unification of the fixated fractured long bone. The healing process of the fractured bone is complicated and delayed union, mal-union and non-union are common occurrences due to the delicate balance between the anabolic and catabolic phases of normal healing. Prior to allowing the patient to return to previous function, the degree of healing is often assessed through clinic interpretation of images from x-rays or CT scans. These radiography techniques are known to be subjective and inconclusive.

The relationship between the state of healing and the increased stiffness of the fractured long bone is widely recognised. A variety of measurement techniques are available, including ultrasound, direct static measurement and vibration measurement, to measure stiffness in an internally fixed fractured bone. Unfortunately, these known techniques all suffer from significant errors and are not suitable for clinical use.

Accordingly, there remains a need to provide a method and system for assessing the state of healing of an internally fixated fractured long bone that ameliorates and/or overcomes disadvantages of known methods and systems for assessing the state of healing of such a bone.

### SUMMARY OF INVENTION

With this in mind, one aspect of the invention provides a method of assessing the state of healing of a fractured long bone in a limb, including the steps of: applying a known force to the limb; using vibration sensors attached on either side of the limb to produce output signals generated in response to the known force from the output signals of the vibration sensors; from the output signals, generating frequency domain waveforms for phase difference between vibration sensor output signals, coherence of the vibration sensor output signals, and cross-spectra of the vibration sensor output signals; identifying in-phase and the out-of-phase responses of the vibration sensors from phase differences in the phase difference waveform at frequencies corresponding to peaks in the cross-spectra waveform; verifying coherent modes from the magnitude of the coherence waveform; and generating bone healing data, including using the magnitude of the coherence waveform and the phase differences as weighting-functions, computing a healing index value representing the state of healing of the bone.

In a method including the steps, the state of healing of fractured long bones can be analysed by measuring bone stiffness through vibrational analysis. The above described steps enable the separation of the transverse and the torsional frequency response to be isolated from the frequency response of a limb to an impact, to thereby enable a better assessment of the state of healing or bone union compared to analysing other response modes.

In one or more embodiments, the step of generating bone healing data further includes generating a first data set over time of healing index values indicative of the progression of the state of healing over time.

In one or more embodiments, the step of generating bone healing data further includes generating a second data set over time of the magnitude of the cross-spectra; and generating a third data set of a time-derivative of the first data set.

In one or more embodiments, the method further includes the step of displaying a visual representation of the bone healing data for interpretation by a clinician.

In one or more embodiments, an internal fixation is applied to the fractured long bone.

The vibration sensors may be radially spaced from each other around the limb by 130 to 240 degrees, and even more preferably by 150 to 210 degrees.

In one or more embodiments, the step of applying an impact to the limb includes causing a mass to travel radially around a limb and strike a strike point fixed to the limb.

Another aspect of the invention provides a system for assessing the state of healing of a fractured long bone in a limb, including: a force application mechanism for applying a known force to the limb; a sensing device for attaching vibration sensors on either side of the limb to produce output signals generated in response to the known force; and a signal analysis arrangement for, from the output signals, generating frequency domain waveforms for phase difference between vibration sensor output signals, coherence of the vibration sensor output signals, and cross-spectra of the vibration sensor output signals; identifying in-phase and the out-of-phase responses of the vibration sensors from phase differences in the phase difference waveform at frequencies corresponding to peaks in the cross-spectra waveform; verifying coherent modes from the magnitude of the coherence waveform; and generating bone healing data, including using the magnitude of the coherence waveform and the phase differences as weighting-functions, computing a healing index value representing the state of healing of the bone.

In one or more embodiments, the signal analysis arrangement is further configured so that generating bone healing data further includes generating a first data set over time of healing index values indicative of the progression of the state of healing over time.

In one or more embodiments, the signal analysis arrangement is further configured so that generating bone healing data further includes generating a second data set over time of the magnitude of the cross-spectra; and generating a third data set of a time-derivative of the first data set.

In one or more embodiments, the system further includes a display for presenting a visual representation of the bone healing data for interpretation by a clinician.

Another aspect of the invention provides a force application mechanism for use in the above-mentioned system, including a mass; a strike point fixed to the limb; and means to cause the mass to travel radially around a limb and strike the strike point.

Yet another aspect of the invention provides an integrated force application mechanism and sensing device for use in the above-mentioned system, including an arrangement for mounting to the limb and integrating (i) the force application mechanism in a housing and (ii) a structure for mounting the vibration sensors on either side of limb.

The invention will now be described in further detail by reference to the accompanying drawings. It is to be understood that the particularity of the drawings does not supersede the generality of the preceding description of the invention.

### BRIEF DESCRIPTION OF DRAWINGS

FIG. 1 is a depiction of two views of a long bone which has been internally fixated with a first plate and screw fixation arrangement;

FIG. 2 is depiction of two views of a long bone which has been internally fixated with an intramedullary nail fixation arrangement;

FIG. 3 is a schematic diagram of a limb around which is attached an arrangement integrating a force application mechanism and vibration sensing device forming part of one embodiment of a system for assessing the state of healing of an internally fixated fractured long bone in a limb;

FIG. 4 is a schematic diagram of the vibration sensing device forming part of the integrated arrangement depicted in FIG. 3;

FIG. 5 is a graphical representation of responses from vibration sensors forming part of the vibration sensing device shown in FIG. 4 to an input torsional load;

FIGS. 65 and 7 are respectively end and isometric views of the force application mechanism forming part of the integrated arrangement shown in FIG. 3;

FIG. 8 is one embodiment of a system for assessing the state of healing of an internally fixated fractured long bone in a limb, in which signal/data processing and information display capability is provided in the integrated arrangement shown in FIG. 3;

FIG. 9 is another embodiment of a system for assessing the state of healing of an internally fixated fractured long bone in a limb, in which data/signal processing and information display is provided separately from the integrated arrangement shown in FIG. 3;

FIG. 10 is a graphical representation of frequency domain waveforms derived from the responses of vibration sensors forming part of the vibration sensing mechanism, the frequency domain waveforms representing the phase difference between vibration sensor outputs, coherence of the vibration sensor outputs and cross-spectra of the vibration sensor outputs; and

FIG. 11 is a graphical representation of two examples of the development of cross-spectra with time and the development of a healing index with time computed from the frequency domain waveforms depicted in FIG. 10.

### DETAILED DESCRIPTION OF PREFERRED EMBODIMENTS

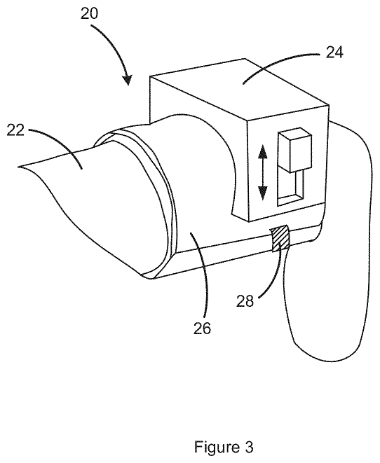

Referring now to FIG. 3, there is shown generally an arrangement 20 mounted to a leg 22 and integrating (i) a force application mechanism in a housing 24 and (ii) a structure 26 for mounting two or more accelerometers or other vibration sensors on either side of the leg 22. Strapping 28 is used to tighten and locate the integrated arrangement 20 to the leg 22. Preferably, the arrangement 20 should be located and fastened around hard-points of the long bone to ensure that the excitation will be applied to the long bone. In the exemplary embodiment shown in FIG. 2, the arrangement 20 is fastened around the epicondyles (hard-points) of the femur.

It will be appreciated that the arrangement depicted in FIG. 3 is merely one convenient manner in which a force application mechanism for applying an impact or other known force to the leg 22 or other limb, and a sensing device for attaching vibration sensors on either side of the limb to produce output signals generated in response to the known force, could be mounted to that limb. In other embodiments, the force application mechanism and sensing device may be formed separately and/or separately attached to the limb.

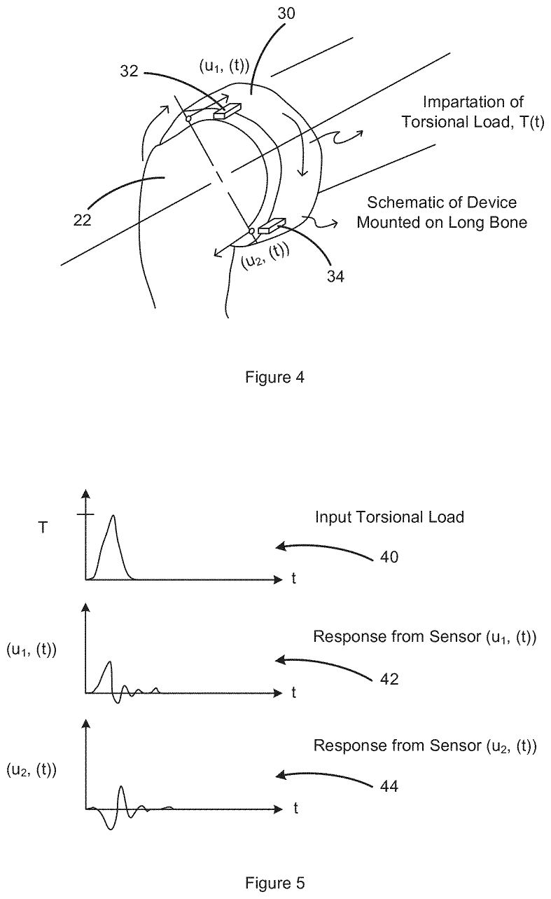

FIG. 4 depicts schematically a portion 30 of the structure 26 which is affixed around the leg 22 to enable accelerometers 32 and 34 to be maintained in position. The accelerometers, or other vibration sensors, are preferably unidirectional and oriented to measure acceleration in the Y-axis direction of the leg 22, that is, parallel to the longitudinal axis of the long bone within the leg 22. The accelerometers are positioned so that when a torsional load is applied to the leg, as shown by the input waveform 40 in FIG. 5, a load is applied to the leg that has a torsional component, the accelerometers 32 and 34 act to generate output signals, respectively referenced 42 and 44, in response to the input load or input load component.

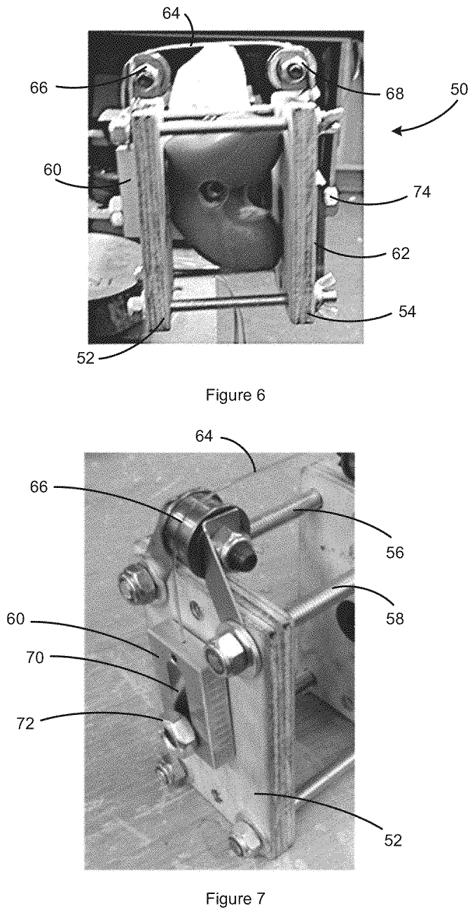

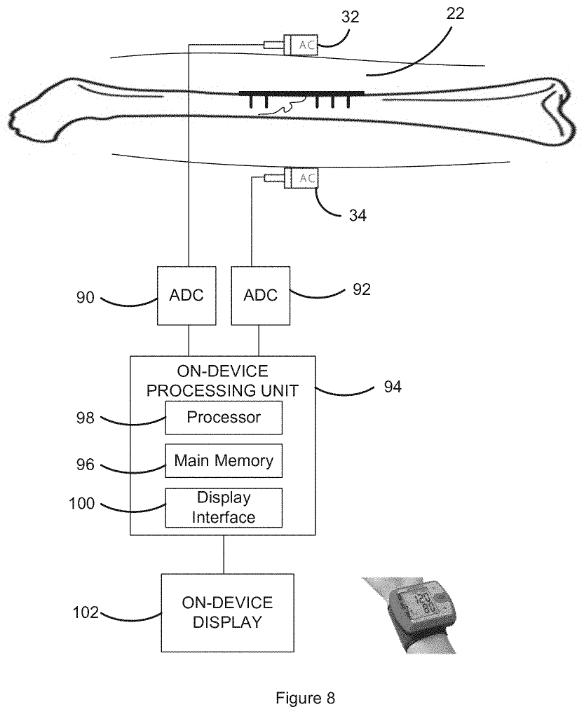

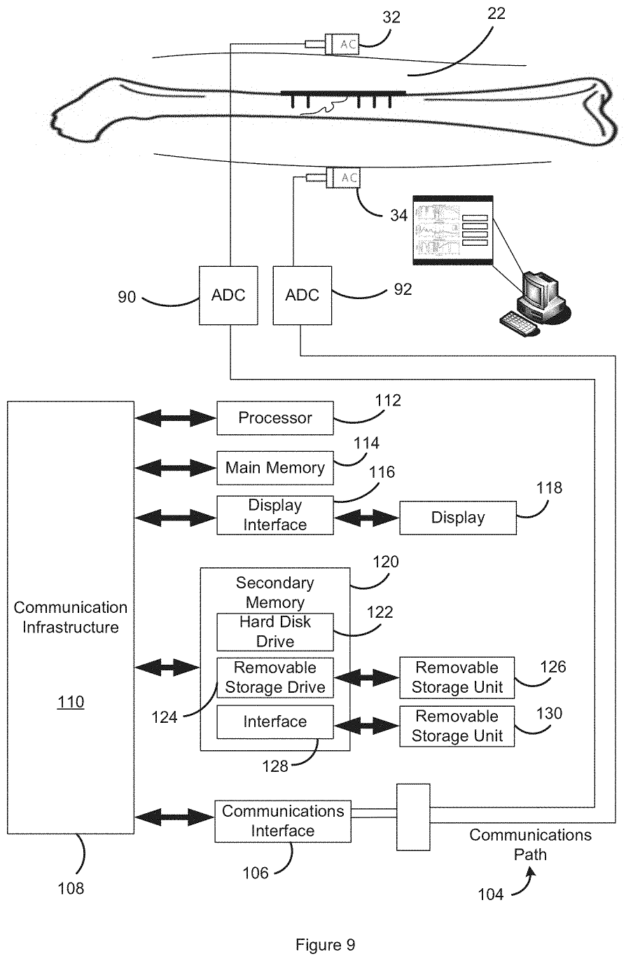

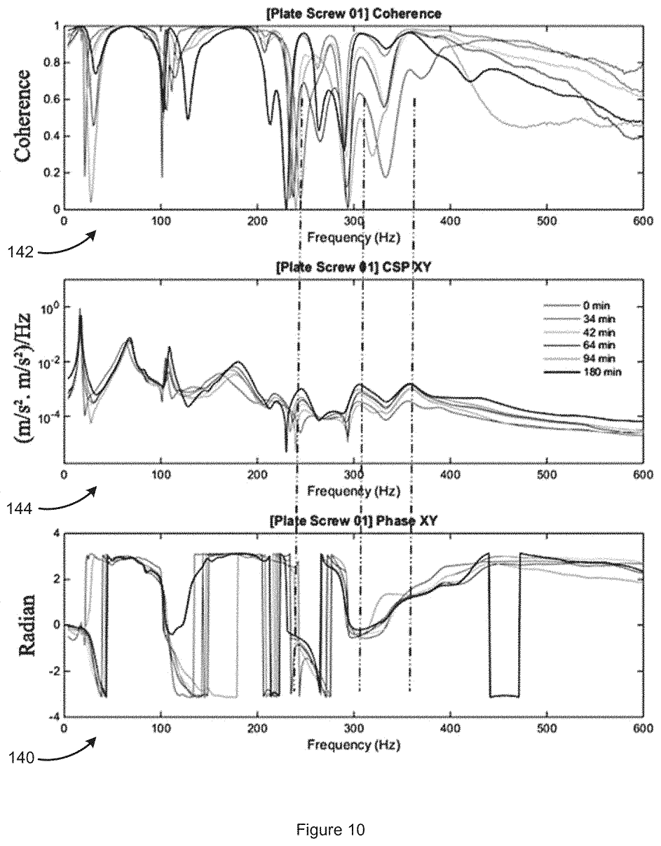

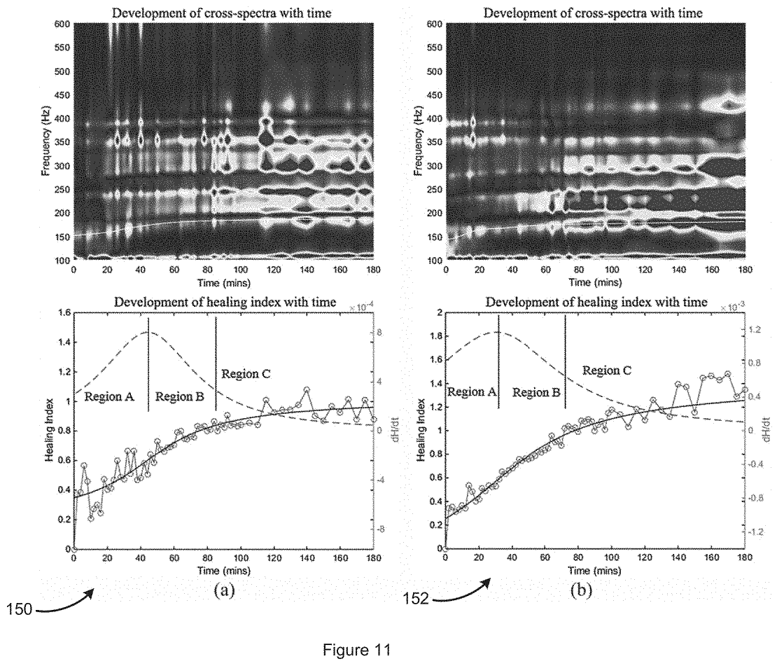

One exemplary arrangement for applying a torsional load to the leg 22 is depicted in FIGS. 6 and 7. The force application mechanism 50 shown in these figures may be housed within the housing 24 shown in FIG. 2. The force application mechanism 50 includes plates 52 and 54 as well as spacing members 56 and 58 acting to separate the plates 52 and 54. Two masses 60 and 62 having different weights are connected by a flexible and stiff cord 64 and suspended over two frictionless bearings 66 and 68.

FIGS. 6 and 7 show the force application mechanism in an “at-rest” state. In order to apply a torsional load to the limb, the mass 60 is pulled down until a limit of movement is reached when one extremity of a slot 70 within the mass 60 impacts a pin 72. In this arrangement, the mass 62 is greater than the mass 60, so upon release of the mass 60, the mass 62 drops due to the differential mass between the two masses 60 and 62. Both masses will travel until the mass 60 strikes the pin 72 at the other extremity of the slot 70, and the mass 62 similarly is caused to strike a pin 74 at the limit of its travel. The impact of the masses on the pins 72 and 74 will subject the force application mechanism 50 and the long bone in the leg 22 to a torsional loading. The impact will give rise to a dynamic loading that will excite the corresponding torsion modes of the long bone Rather than rely upon gravity, other embodiments of the invention may rely upon solenoids or other electromagnetic means to cause the masses to strike the pins and deliver a torsional load to the limb.

In embodiments of the invention in which the force application mechanism is not mounted within the housing 24, but rather separately attached to the leg 22, tensioning devices such as the arrangement of nuts and bolts shown in FIGS. 6 and 7 may be used to clamp the two plates 52 and 54 to the leg 22.

Whilst it is preferable to apply a torsional load to the limb, that is, a force that is applied around the longitudinal axis of the limb, in other embodiments of the invention may the force application mechanism apply the load/force in a different direction. Yet other embodiments of the invention may omit a force application mechanism altogether, and other means be used to apply a load/force to the leg, such as a conventional physician's hammer.

Referring now to FIG. 8, the output signals generated by each of the accelerometers 32 and 34 are then supplied to analogue to digital converters, respectively referenced 90 and 92 to enable downstream signal processing. In the embodiment shown in FIG. 8, the data/signal processing is carried out by a processing unit 94 mounted to or forming part of the integrated arrangement 20 in FIG. 3. As is conventionally the case, the processing unit 94 includes a main memory 96 for storing program instructions and a processor 98 for performing the various data processing and other operations required to be performed. A display interface 100 is also provided to enable feedback and an indication of the state of healing of the bone 22 to be provided to a user at an on-device display 102.

In another embodiment of the invention depicted in FIG. 9, the data/signal processing of the output signals produced by the analogue to digital converters 32 and 34 is carried out remote from or separate to the integrated arrangement 20 encasing the leg 22 or other limb. In such an embodiment, the digitised signals from the accelerometers 32 and 34 that are provided by the analogue to digital converters 90 and 92, are supplied by a communications path 104 to the communications interface 106 of a computing system 108. The computing system 108 includes a communication infrastructure 110 enabling communication to occur between a processor 112, main memory 114 and display interface 116 enabling user feedback via a display 118. The main memory 114 stores program instructions to cause the processor 112 to carry out designed and programmed functionality.

In addition, a secondary memory 120 may be provided including such data storage devices as a hard disk drive 122, a removable storage drive 124 for storing a removable storage unit 126 and an interface 128 for interacting with a second removable storage unit 130. Whilst the processing power and size of the display of the arrangement shown in FIG. 8 will necessarily limit feedback information provided to a user or medical practitioner, an off-device arrangement of the type shown in FIG. 9 will provide greater processing power and the ability to provide richer graphical and other diagnostic information to a user or medical practitioner.

The accelerometers 30 and 32 respond in phase, when detecting a translational response to the impact applied to the leg 22, or out-of-phase when detecting a torsional response. The digitised signals generated by the analogue to digital converters 90 to 92 are analysed to isolate the torsional modes and/or bending modes from the recorded frequency response of the accelerometers. Analysing the torsional frequency response in isolation yields a better assessment of the state of healing or bone union compared to analysing other response modes.

After the torsional modes and/or bending modes are isolated, they can be mapped to a healing index providing an indication of the state of healing of the bone within the leg 22. Referring now to FIG. 10, there is shown respectively frequency domain waveforms for phase difference 140 between vibration sensor output signals, coherence 142 of the vibration sensor output signals and cross-spectra 144 of the vibration sensor output signals.

The in-phase and out-of-phase responses of the vibration sensors are identified by the data/signal process unit 94 or computing system 108 from phase differences in the phase difference waveform at frequencies corresponding to peaks in the cross-spectra waveform. Coherent modes are then verified from the magnitude of the coherence waveform. Finally, using the magnitude of the coherence waveform and the phase differences as waking functions, a healing index value representing the state of healing of the bone is computed and displayed to the user.

FIG. 10 also shows the variation of the measured dependent variables as a function of time (i.e., different stages of healing). The exact form of the waveforms 140 to 144 have been generated using an experimental setup in which a composite femur was fixated with a model T2IM nail from Striker Corporation. In this arrangement, the head of the femur was fastened with a vice rigidly attached to a heavy block of concrete. The femur was securely gripped with a set of 3D-printed vice clamps matched to the femur head geometry. Two unidirectional accelerometers (B&K Type 4507), which have been orientated to measure acceleration in the Y-axis direction, were attached to the test specimen. A saw blade was used to perform a mid-shaft osteotomy of the composite femur and an intramedullary retrograde femur nail was inserted via a distal entry point, and cross bolts were inserted at distal and proximal ends. A tape was placed over the fractured region to form a mould which was filled with the epoxy adhesive. At this point, modelling clay was added to the femur to facilitate the observation of the healing process, a two-part epoxy with a curing time of 30 min (8 h to achieve full strength) was then prepared and then filled into the osteotomised region. The mass of the modelling clay used was 1 kg. The final mass of the composite femur, fixation, and modelling clay are tabulated in Table 1.

TABLE 1
Mass of test specimen with IM Nail
fixation. [AQ: 3]

| | |
|---|---|
| Sawbone femur | 512 g |
| Sawbone femur + fixation | 638 g |
| Sawbone femur + fixation + clay | 1638 g |

The chemical reaction begins upon mixing of the two-part epoxy. This means that the epoxy will cure as the test specimen is being prepared, which includes the installation of the modelling clay. Time t=0 s will denote the first set of experimental results recorded. Subsequent experiments were conducted at regular intervals as the epoxy cures and ‘heals’ in the osteotomised region of the femur. The experiments were conducted for up to 180 min after mixing the epoxy in order to span the entire curing process.

Although the test equipment has an anti-aliasing function, the measurement oversampled at a sampling rate of 22,000 samples per second (bandwidth of 10 kHz), with a frequency resolution of 1.56 Hz. Each spectrum was averaged over 10 samples. The expected useful bandwidth is 600 Hz. The oversampling adopted will eliminate the potential of aliasing. This number of samples provided a good signal-to-noise ratio, and the spectra were observed to stabilise after averaging 7 samples. The measurement at each state of healing takes approximately 30 s, which is not significant compared with the curing time of the adhesive.

The dependent variables used to characterise the dynamic response of the fixated femur include the magnitude and phase of the cross-spectrum and the coherence function calculated from the two-sensor arrangement. These quantities are plotted as a function time which is then used to represent the independent variable, ‘simulated healing time’. The cross-spectrum between accelerometers S1 and S2 and the coherence function from the two accelerometers were determined at 2 min intervals for the first 100 min and 5 min intervals afterwards. The coherence function underpinned the statistical significance of the measured signals (S1 and S2).

Attempts had been made to control other factors influence, such as the volume of epoxy filled in the osteotomised region of the composite femur and the specimen preparation time. The specimen preparation time includes the duration of mixing the two-part epoxy, filling the osteotomised region and wrapping the femur with the modelling clay. The variations due to these factors are likely to affect the results and constitute a good experimental test for the efficacy and the veracity of the statement that the dynamic response of the fixated femur is a useful and robust method for assessing the state of healing of the fractured region. In spite of these, the results will show that the state of healing can be assessed from the dependent variables measured.

It will be appreciated that this experimental setup simulates a fractured and internally fixated femur in a leg, and confirms through a simulated and accelerated healing process the functionality of the present invention. However, it will be appreciated that the exact nature of the waveforms and their change or evolution over time as healing occurs will differ when the invention is applied to a real limb and will also differ as a function of the particular long bone that is to be assessed.

The coherence function between accelerometers 32 and 34 determines the causality between these accelerometers and identifies coherent mode frequencies. The effects of the modelling mass are not evident at frequencies below 100 Hz. The response within this frequency bandwidth is associated with the global response of the construct. However, the effects of mass loading imposed by the modelling clay are evident at higher frequencies. The first out-of-phase mode of the construct with and without ‘mass loading’, with significant coherence was observed in the proximity of 285 and 305 Hz, respectively. The ‘in-phase’ mode with and without ‘mass loading’ with significant coherence was measured at approximately 250 and 370 Hz, respectively. In addition, the inclusion of the modelling clay suppressed the magnitude of the cross-spectral. This is attributed to the effects of damping of the modelling clay, which acts to simulate tissue surrounding the fractured bone.

As mentioned above, FIG. 10 shows a variation of the measured dependent variables as a function of time (i.e. different stages of simulated healing). The main observations of the results are as follows:

(a) The in-phase modes are observed at 16, 109 and 240 Hz.
The coherence at first in-phase mode at 16 Hz is close to unity from the start of the experiment and was not observed to change as a function of the state of healing. The dynamic stiffness associated with this mode is not sensitive to the state of healing.
The frequency associated with the peak of the second in-phase mode was observed to increase with time of healing. This is attributed to the increased in stiffness resulting from the curing (i.e. simulated healing) of the fixated femur.
The appearance of the third in-phase mode in the vicinity of 245 Hz clearly demonstrated the viability of using the change in the dynamic stiffness to account for the state of healing of the fixated fracture femur. Initially, the magnitude of the coherence function at this frequency was low, and increased close to unity towards the conclusion of the experiment (i.e. with progression of simulated healing).
   (b) The out-of-phase (OOP) modes were observed to behave as follows:
The first OOP mode at 61 Hz has a coherence value close to unity throughout the experiment and is associated with the global mode of the construct. The increase in the dynamic stiffness associated with this mode as a function of healing is not significant.
The second OOP mode is observed to initially develop at 143 Hz and then migrated to 190 Hz towards the end of the experiment. The development of this mode is substantiated with the corresponding magnitude of the coherence function measured. This observation is consistent with the increased stiffness associated with the progression in simulated healing at the fractured region.
The third OOP mode is observed in the vicinity of 210 Hz. The definition of this mode improves as a function of time (i.e. healing). The increasing magnitude of the coherence function towards unity at the frequency is consistent with the healing progression of the fractured region.

After at a healing index value representing the state of healing of the bone can then be computed using the magnitude of the coherence waveform and the phase differences as waking functions, in accordance with the normalised healing index, for example, as defined in equation 1. Equation 1 is one example of a function that estimates the state of healing from the dynamic response of the structure. The Healing Index as defined in equation 1 increases monotonically and asymptote as healing progresses.

$$\begin{matrix}HI ( t ) = \frac{1}{Initial Heating Index} {\int }_{0}^{600} \vert CS ( f , T = t ) - CS ( f , t = 0 ) \vert df \\ where Initial Healing Index = {\int }_{0}^{600} CS ( f , t = 0 ) df & ( 1 ) \\ {HI}_{i} ( t ) = \frac{d}{dt} ( HI ( t ) ) & ( 2 )\end{matrix}$$

The frequency bandwidth between 0 and 600 Hz is chosen to include the modes sensitive to healing. After integration, the index is normalised to the cross-spectrum at time zero (equation (1)). FIGS. 11(a) and (b) shows the application of equation (1) to the two sets of experimental results described above. In spite of the presence of mass loading, this plot indicates that a healed femur will return a significant value for healing index compared with that for a fractured fixated femur. The healing index curve shows the progression of healing and asymptotes with increasing time.

The time-derivative of the normalised healing index (HIₜ), as calculated by equation (2) and the evolution of the cross-spectra with respect to the healing of the femur are presented in graphs 150 and 152 in FIGS. 11(a) and (b). The magnitudes of the cross-spectra measured during the experiment are presented in this intensity plot. The regions A, B and C show the behaviour of the healing index curve used for healing assessment. The healing assessment is best conducted by considering the magnitude of the cross-spectrum, the healing index curve and the rate of the change of the healing index simultaneously.

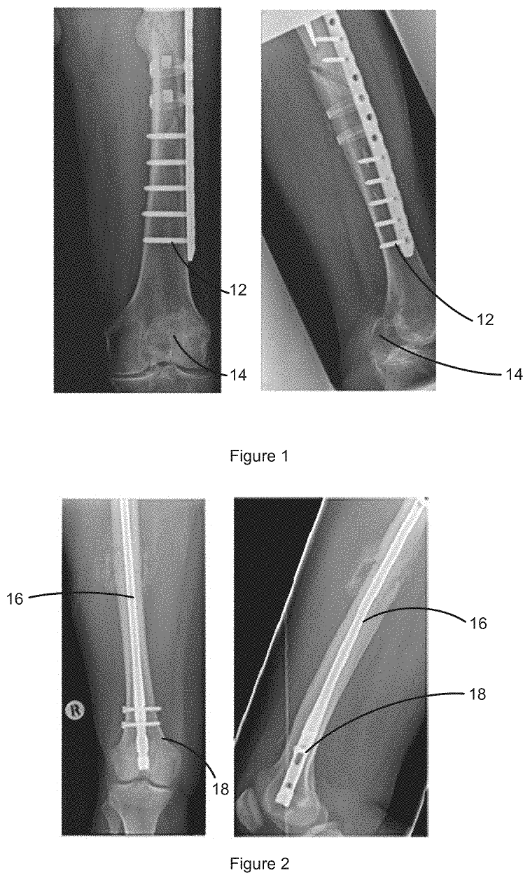

In Region A, the start of the healing will give rise to an increase in the healing index, and this is accompanied by the change in the cross-spectrum that alludes to an increase in the stiffness of the entire construct. The increase in stiffness is evident in the cross-spectra curves. A curve is plotted along the peaks on the cross-spectra plots in FIGS. 1(a) and (b) to show the stiffness increment as healing progressed. Region B is associated with the decelerating rate of healing that eventually asymptotes (Region C). The asymptotic behaviour of the healing index is associated with the formation of the higher modes that arises due to the later stages of healing of the fractured region.

Whilst the invention has been described in conjunction with the limited number of embodiments, it will be appreciated by those in the art that many alternatives, modifications and variations are possible in light of the foregoing description. The present invention is intended to embrace all such alternatives, modifications and variations as may fall within the spirit and scope of the invention as disclosed.

1. A method of assessing the state of healing of a fractured long bone in a limb, including the steps of:
applying a known force to the limb;
using vibration sensors attached on either side of the limb to produce output signals generated in response to the known force from the output signals of the vibration sensors;
from the output signals, generating frequency domain waveforms for phase difference between vibration sensor output signals,
coherence of the vibration sensor output signals, and
cross-spectra of the vibration sensor output signals;
identifying in-phase and the out-of-phase responses of the vibration sensors from phase differences in the phase difference waveform at frequencies corresponding to peaks in the cross-spectra waveform;
verifying coherent modes from the magnitude of the coherence waveform; and
generating bone healing data, including
using the magnitude of the coherence waveform and the phase differences as weighting-functions, computing a healing index value representing the state of healing of the bone.

2. The method according to claim 1, wherein the step of generating bone healing data further comprises:
generating a first data set over time of healing index values indicative of the progression of the state of healing over time.

3. The method according to claim 2, and wherein the step of generating bone healing data further comprises:
generating a second data set over time of the magnitude of the cross-spectra; and
generating a third data set of a time-derivative of the first data set.

4. The method according to claim 1, further comprising:
displaying a visual representation of the bone healing data for interpretation by a clinician.

5. The method according to claim 1, wherein an internal fixation is applied to the fractured long bone.

6. The method according to claim 1, wherein the vibration sensors are radially spaced from each other around the limb by 130 to 240 degrees.

7. The method according to claim 6, wherein the vibration sensors are radially spaced from each other around the limb by 150 to 210 degrees.

8. The method according claim 1, wherein the step of applying a known force to the limb includes causing a mass to travel radially around a limb and strike a strike point fixed to the limb.

9. A system for assessing the state of healing of a fractured long bone in a limb, including:
a force application mechanism for applying a known three to the limb;
a sensing device for attaching vibration sensors on either side of the limb to produce output signals generated in response to the known force; and
a signal analysis arrangement configured to generate frequency domain waveforms from the output signals for
a) phase difference between vibration sensor output signals,
b) coherence of the vibration sensor output signals, and
c) cross-spectra of the vibration sensor output signals;
identifying in-phase and the out-of-phase responses of the vibration sensors from phase differences in the phase difference waveform at frequencies corresponding to peaks in the cross-spectra waveform;
verifying coherent modes from the magnitude of the coherence waveform; and
generating bone healing data, including
using the magnitude of the coherence waveform and the phase differences as weighting-functions, computing a healing index value representing the state of healing of the bone.

10. The system according to claim 9, wherein the signal analysis arrangement is further configured so that generating bone healing data further comprises:
generating a first data set over time of healing index values indicative of the progression of the state of healing over time.

11. The system according to claim 10, wherein the signal analysis arrangement is further configured so that generating bone healing data further comprises:
generating a second data set over time of the magnitude of the cross-spectra; and
generating a third data set of a time-derivative of the first data set.

12. The system according to claim 9, wherein an internal fixation is applied to the fractured long bone.

13. The system according to claim 9, further comprising:
a display for presenting a visual representation of the bone healing data for interpretation by a clinician.

14. The system according to claim 9, wherein the vibration sensors are radially spaced from each other around the limb by 130 to 240 degrees.

15. The system according to claim 14, wherein the vibration sensors are radially spaced from each other around the limb by 150 to 210 degrees.

16. A force application mechanism for use in a system according to claim 9, comprising:
a mass;
a strike point fixed to the limb; and
means to cause the mass to travel radially around a limb and strike the strike point.

17. An integrated force application mechanism and sensing device for use in a system according to claim 9, comprising:
an arrangement for mounting to the limb and integrating (i) the force application mechanism in a housing and (ii) a structure for mounting the vibration sensors on either side of limb.
